(19) United States
(12) Patent Application Publication
(10) Pub. No.: US 2020/0295768 A1
(43) Pub. Date: Sep. 17, 2020

(54) CRYSTAL-FREE OSCILLATOR FOR CHANNEL-BASED HIGH-FREQUENCY RADIO COMMUNICATION

(71) Applicants: Novo Nordisk A/S, Bagsvaerd (DK); Polaric Semiconductor IVS, Koebenhavn SV (DK)

(72) Inventors: Carsten Bleser Rasmussen, Kiev (UA); Ching-Hua Yang, Hsinchu County 30345 (TW); Ander Fernandez Garcia, Alava (ES); Mikkel Schouenborg Grubbe, Hilleroed (DK)

(21) Appl. No.: 16/768,276

(22) PCT Filed: Nov. 30, 2018
(86) PCT No.: PCT/EP2018/083170

§ 371 Date: May 29, 2020

(30) Foreign Application Priority Data
Dec. 1, 2017 (EP) 17204950.4

(51) Int. Cl. H03L 7/099; H03L 1/02; A61M 5/24; A61M 5/50

(52) U.S. Cl. CPC H03L 7/099; H03L 1/02; A61M 5/24; A61M 5/5086; A61M 2205/3569; A61M 2205/3592; A61M 2205/3584; A61M 2205/3553; A61M 2205/3561; A61M 2205/3368

(57) ABSTRACT

The present invention relates to a crystal-free oscillator circuit (100) for channel-based high-frequency radio communication, the crystal-free oscillator circuit (100) comprising a crystal-free oscillator element (120) configured to provide a high-frequency reference signal (101), the high-frequency reference signal (101) having a frequency of at least about 1 GHz, and a phase-locked loop (PLL) circuit (110) having a feedback loop and comprising a PLL oscillator (120), wherein the phase-locked loop circuit (110) is configured to receive a high-frequency reference signal (101), to provide a feedback signal (102) in the feedback loop, and to provide a high-frequency output signal (103), the high-frequency output signal (103) being generated by the PLL oscillator (120′) in response to the high-frequency reference signal (101) and to the feedback signal (102) where the feedback signal (102) is dependent on an earlier instance of the output signal (103), wherein the crystal-free oscillator circuit (100) further comprises an adjustable frequency offset circuit (210) located in the feedback loop, the adjustable frequency offset circuit (210) comprising a frequency generator (200) and being configured to offset a frequency of the feedback signal (102) in response to an adjustment control signal (104), and wherein the crystal-free oscillator circuit (100) is configured to compensate for a temperature dependency of the crystal-free oscillator circuit (100) in response to a measured current operating temperature.

### FIELD OF THE INVENTION

The invention generally relates to crystal-free oscillator circuit for channel-based high-frequency radio communication, the crystal-free oscillator circuit comprising a crystal-free oscillator element configured to provide a high-frequency reference signal, the high-frequency reference signal having a frequency of at least about 1 GHz and a phase-locked loop circuit having a feedback loop and comprising a voltage controlled oscillator, where the phase-locked loop circuit is configured to receive the reference signal, to provide a feedback signal in the feedback loop, and to provide a high-frequency output signal, where the high-frequency output signal is derived by the voltage controlled oscillator in response to the high-frequency reference signal and to the feedback signal.

### BACKGROUND

Many types of wireless radio-based communications systems with channel synthesis, i.e. channel-based radio communication, include the use of a crystal-based oscillator as a high-frequency reference as generally known.

Such crystal-based oscillators typically have an active part that may be manufactured as an integrated circuit or ‘on-chip’ and an external resonator part comprising the crystal. They have many advantageous characteristics including generally being insensitive in relation to operating temperature, having low phase noise and high-frequency accuracy in general and e.g. when used together with frequency dividers and/or other elements, etc.

These advantageous characteristics of crystal-based oscillators have made them basically a first choice for use in many types of communications systems and in particular for channel-based radio communications systems, which generally are sensitive in relation to phase noise.

However, the costs for such crystals and crystal-based oscillators with required specs and tolerances for use with channel-based radio communications systems are relatively high due to not being monolithic (i.e. they comprise distinct separate parts that cannot be implemented by a single integrated circuit; namely the active silicon part and the external resonator part with a quartz crystal), due to the crystal needing to be in a vacuum casing, etc. Therefore, it is not generally feasible to use such crystal-based communications systems for more or less disposable products e.g. involving only a single use, a few uses, or uses only for a limited amount of time such as for about a month or couple of weeks or less.

Certain crystal-free oscillators of various types are generally known, e.g. LC oscillators, ring oscillators, RC oscillators, etc., that are cheaper to produce compared to crystal-based oscillators, at least in part due to being monolithic. Crystal-free oscillators may e.g. be manufactured as a solid-state integrated circuit having a resonator element or circuit.

However, such crystal-free oscillators are not directly and immediately suitable or even usable for use in channel-based high-frequency radio communication systems due to certain drawbacks (e.g. when compared to crystal-based oscillators) including generally having a relatively low q-factor, higher manufacturing variation resulting in parameter variation from oscillator to oscillator (even when produced on a same wafer or similar e.g. due to so-called on-chip variation (OCV)), operating temperature sensitivity potentially leading to reduced frequency accuracy and/or increased phase noise during operation (e.g. resulting in communications errors, dropping of a channel, degraded/un-reliable communication, and so on), etc.

When used in radio communication, such crystal-free oscillators are typically used together with integer frequency dividers, fractional N dividers, or the like in a phase-locked loop (PLL) circuit or other as generally known to reduce the frequency of the oscillator element or circuit to a frequency usable for the radio communication according to relevant standards and specifications. As an example, Bluetooth and Bluetooth Low Energy (BLE) equipment operates at frequencies between 2402 and 2480 MHz (with 79 1-MHz channels for Bluetooth and 40 2-MHz channels for BLE).

On one hand, having a crystal-free oscillator with a relatively high frequency (before being divided down) is generally an advantage in relation to channel-based radio communication since it reduces phase noise impacting signal to noise ratio and bit error rate (BER). On the other hand, dividing down a relatively high frequency of a crystal-free oscillator by a large multiple (e.g. by 1000, 100, or even 10, or multiples thereof) introduces phase noise. Generally, dividing a frequency in the present context by a multiple of 2 introduces about 3 dB of noise. Needing an output frequency of 1 MHz and having a crystal-free oscillator operating at 2 GHz (as an example) would involve dividing by 2000 and introduce about 32 dB of phase noise due only to the division.

Therefore, traditional crystal-free oscillators will have a relatively high (and generally too high) phase noise/bit error rate for channel-based radio communication, especially when used together with integer frequency dividers, etc.

Using so-called fractional N dividers instead of integer dividers may mitigate certain aspects, but then generally introduce (too much) jitter noise making also them not immediately suitable or even usable in connection with crystal-free oscillators used for channel-based radio communication.

Other types of crystal-free oscillators include MEMS (Micro-Electro-Mechanical Systems) and other mechanically based oscillators. However, such mechanically based oscillators have a drawback of e.g. not being able to be miniaturised sufficiently for many uses. Additionally, the etching process and production time for MEMS is still relatively time consuming. Combining MEMS and radio frequency (RF) modules as an integrated circuit package is difficult and costly. Furthermore, it is complex and costly to obtain sufficient performance for both MEMS and RF of a circuit at the same time.

Patent specification U.S. Pat. No. 5,604,468 discloses a frequency synthesizer with temperature compensation and frequency multiplication. The frequency synthesizer comprises a crystal-based temperature dependent frequency oscillator to generate a reference signal that is provided to a PLL circuit preferably via a temperature-independent divider. The PLL circuit comprises a dual-modulus fractional N divider, where N preferably is 100/101, that is controlled by a temperature compensation control circuit providing a temperature-dependent modulator control signal along with a desired PLL multiplication factor to the dual-modulus fractional N divider. Fractional N dividers can be used to good effect when used in connection with a good quality oscillator, such as at least some crystal-based oscillators. However, as mentioned, using a fractional N divider in a PLL circuit introduces (too much) jitter noise when used with low quality crystal-based oscillators or when used with crystal-less oscillators making them in such configurations unsuitable for channel-based high-frequency radio communication; at least without adding further appropriate circuitry addressing this, which would increase cost, complexity and not the least power consumption. Additionally, dividing by a relatively large integer (such as 100 or 101) introduces (too much) phase noise when used with low quality crystal-based oscillators or with crystal-less oscillators making such circuits unsuitable for channel-based high-frequency radio communication. Again this would need to be addressed, adding costs, complexity, and power consumption.

Patent specification US 2007/0176690 discloses an integrated circuit comprising a crystal oscillator emulator where the integrated circuit includes a microelectromechanical (MEMS) or film bulk acoustic resonator (FBAR) resonator circuit that generates a reference frequency, i.e. mechanical oscillators. The integrated circuit further comprises a fractional phase locked loop with a temperature compensation input. The circuit comprises a voltage controlled oscillator (VCO) generating a VCO output that is fed back to a fractional divider dividing the VCO frequency by N or N+1 by a scaling circuit. Calibration information is obtained in response to a temperature signal where the calibration information adjusts a ratio of the divisors N and N+1 that are used by the scaling circuit. The integrated circuit according this disclosure has the same disadvantages as mentioned for the disclosure above.

Patent specification WO 2006/000611 discloses a method of stabilising a frequency of a frequency synthesizer using a mechanical oscillator in the form of a MEMS reference oscillator coupled to a DLL that also involves use of a divider in the form of a fractional-N divider. This disclosure has the same disadvantages as mentioned for the disclosures above.

It would accordingly be a benefit to have a crystal-free oscillator being suitable for use in channel-based high-frequency radio communication systems, and in particular a crystal-free oscillator that would adhere to required tolerances for channel-based high-frequency radio communications. Furthermore, a crystal-free oscillator that is (relatively) cheaper to manufacture would also enable wireless/radio-based high-frequency communications applications even for relatively low-cost, single-use/few-uses, and/or time-limited products.

### SUMMARY

It is an object to alleviate at least one or more of the above mentioned drawbacks at least to an extent.

An aspect of the invention is defined in claim 1.

Accordingly, in one aspect of the present invention, a crystal-free oscillator circuit for or configured to provide channel-based high-frequency radio communication is provided where the crystal-free oscillator circuit comprises a crystal-free oscillator element configured to provide a high-frequency reference signal, where the high-frequency reference signal has a frequency of at least about 1 GHz (e.g. at least 1 GHz). In some embodiments, the high-frequency reference signal has a frequency of at least about 2 GHz (e.g. at least 2 GHz) and/or as disclosed herein. Such a relatively high operating frequency (frequency of the reference signal) is advantageous as it reduces or minimizes phase noise and improves the q-factor (compared to a crystal-free oscillator element operating at lower frequency) being quite significant for channel-based high-frequency radio communication. Preferably, the crystal-free oscillator element is an LC-based oscillator. The crystal-free oscillator circuit further comprises a phase-locked loop (PLL) circuit having a feedback loop and comprising a PLL oscillator (also crystal-free). The PLL is preferably a high-speed PLL, i.e. operating at a frequency of at least 1 GHz or at least 2 GHZ. The operating frequency should be sufficiently high to enable a monolithic implementation and sufficiently high to enable the output to be used in connection with a carrier frequency of a relevant channel-based high-frequency radio communication. The crystal-free oscillator element is a non-mechanical oscillator. For an analog crystal-free oscillator circuit, the PLL oscillator may e.g. be a voltage controlled oscillator (VCO). The phase-locked loop circuit is configured to receive the high-frequency reference signal (from the crystal-free oscillator element or circuit), to provide a feedback signal in the feedback loop, and to provide a high-frequency output signal. The high-frequency output signal is generated by the PLL oscillator in response to the high-frequency reference signal and in response to the feedback signal where the feedback signal is dependent on an earlier instance of the high-frequency output signal. It is noted that the term “feedback signal” herein designates the signal in the whole feedback loop even though the feedback signal will be modified, processed, changed, etc. by various elements as disclosed herein. The crystal-free oscillator circuit further comprises an adjustable frequency offset (e.g. digital) circuit located in the feedback loop. The adjustable frequency offset circuit comprises a frequency generator or the like where the adjustable frequency offset circuit is configured to offset (at least during use at an appropriate operating or sample frequency for digital elements or continuously for analog elements) a frequency of the feedback signal in response to an adjustment control signal. The adjustment control signal may e.g. be supplied by a processing element or circuit (e.g. also providing controls signals as disclosed herein) connected to a memory. The frequency generator is configured to generate a periodic signal having a frequency set under the control of the adjustable frequency offset circuit. In some embodiments, the frequency of the periodic signal being generated by the frequency generator is set in dependency of the adjustment control signal (e.g. set directly in response to the adjustment control signal or set indirectly in response to the adjustments control signal, i.e. in response to a modified or processed adjustment control signal). The frequency offset may in particular e.g. further relate to one or more channel settings (e.g. changing of), modulation, etc. of the channel-based high-frequency radio communication. The appropriate operating or sample frequency may e.g. depend on a gradient of a temperature sensitivity or tolerance (e.g. in a temperature operation range) of the crystal-free oscillator circuit and/or the crystal-free oscillator element. Preferably (but not necessarily), the crystal-free oscillator circuit further comprises a first static frequency divider located in the feedback loop and being configured to divide down a frequency of the feedback signal by a factor being a first predetermined positive integer (N). In some embodiments, N is relatively small, i.e. less than 10 or more preferably equal to or less than 6 or 4 ensuring that phase noise introduced by division does not become too large for channel-based high-frequency radio communication. In some embodiments, N is 2. It is noted, that the frequency offset is not performed by changing the factor (N); therefore the designation ‘static’ frequency divider. During operation, the first (static) frequency divider will always divide down by the first predetermined positive integer (N) being a constant.

Offsetting a frequency in the feedback loop is different than adjusting the frequency using fractional dividers or fractional-N dividers dividing down to a large extent (e.g. often dividing down by a factor being larger than 64 or even 128 or more). By offsetting a frequency in the feedback loop comprising a (static) divider, the noise is added/additive for relatively small offset values (small relative to the frequency of the feedback signal) instead of being multiplied/multiplicative as e.g. is the case when using controlled fractional dividers or fractional-N dividers in the feedback loop together with a low-quality crystal-based oscillator or with crystal-free oscillators without any of the effects of the present invention. Applying an offset in the disclosed way does not degenerate the noise performance of the PLL oscillator as otherwise would be the case for other crystal-free solutions. Using an offset also enables very fine tuning of the frequency. Offsetting by the adjustable frequency offset circuit enables use of a high frequency feedback signal without introducing (too much) phase noise and/or jitter thereby making it usable for channel-based high-frequency radio communication. Additionally, the crystal-free oscillator circuit is configured to compensate for a temperature dependency of the crystal-free oscillator circuit in response to a measured current operating temperature. This temperature dependency compensation is in some embodiments done by adjusting signals (in particular adjusting the output frequency of the crystal-free oscillator element as disclosed herein) or other aspects of the crystal-free oscillator element. Alternatively, the temperature dependency compensation is done by the adjustable frequency offset circuit, whereby the needed temperature compensation data is or may be included as part of the adjustment control signal. Having the adjustable frequency offset circuit compensating for the temperature dependency is generally more precise since it generally is less sensitive to process variations, while temperature compensating via the crystal-free oscillator element generally can compensate for higher variations. The adjustment control signal may be provided to the adjustable frequency offset circuit externally or alternatively the adjustable frequency offset circuit may be configured to generate the adjustment control signal (in such cases then e.g. receiving a signal representing a current operating temperature of the crystal-free oscillator circuit or a part thereof).

By having an adjustable frequency offset circuit ongoingly offset (at least during operation/use) the input (by offsetting the feedback signal) of the PLL circuit thereby offsetting the whole PLL circuit or significant elements thereof, the PLL circuit becomes trimmable with respect to the operating frequency. This enables ongoing modulation or tuning of the PLL circuit, and more particularly ongoing modulation or tuning of the operating frequency. This in turn enables ongoing compensation for the otherwise inherent frequency related or frequency influencing drawbacks of a crystal-free oscillator element (and thereby of the whole crystal-free oscillator circuit). In particular, it is enabled to use a crystal-free oscillator in channel-based high-frequency radio communication systems as the phase noise can be adjusted and controlled to be within required or preferred specifications and/or tolerances (by not having to divide down to a large extent). This also enables compensating for process, voltage, and temperature (PVT) variation effects.

For channel-based radio communication involving a linear phase, e.g. such as Bluetooth Low Energy (BLE), there will be a constant amplitude involved and there is no need for an AM part. Accordingly, it is possible to avoid having to use quadrature upconverters/modulators (IQ modulators) as otherwise traditionally are used e.g. in BLE for modulating quadrature amplitude modulation (QAM) if the PLL circuit as disclosed herein is used (as part of a transmitters) for phase and/or frequency modulation (without AM). Avoiding such a quadrature upconverter/modulator (IQ modulator) (even/also for BLE) will reduce the complexity of the overall circuit(s) and power usage during operation.

It is an advantage to trim (i.e. offset the frequency) in the feedback loop, especially if the feedback loop comprises at least one frequency divider, since the noise in this way is added/additive instead of being multiplied/multiplicative as e.g. is the case for certain prior art circuits.

In some embodiments, the crystal-free oscillator element is an LC-based oscillator (LCO). An advantage of an LC-based crystal-free oscillator element is e.g. that it readily enables sufficiently high frequencies (e.g. about 1 GHz or more, about 2 GHz or more, etc.) and in particular frequencies usable for channel-based high-frequency radio communication.

In some further embodiments, the LC-based oscillator (LCO) comprises a fixed inductor part and a controllable and variable capacitor part, wherein the controllable and variable capacitor part comprises at least one fixed or base capacitor and one or more of: a group of switchable capacitors (controlled in response to a first tuning control signal) and at least one voltage controlled capacitor (controlled in response to a second tuning control signal), wherein the LC-based oscillator (LCO) is configured to be temperature compensated by adjusting an output frequency of the LC-based oscillator (LCO) in according with the first tuning control signal and/or the second tuning control signal provided in response to a temperature sensor signal provided by a temperature sensor located in the vicinity of the LC-based oscillator (LCO).

In this way, the frequency of the reference signal (as output by the LC-based oscillator (LCO) may be adjusted to compensate for temperature dependency by providing appropriate (first and/or second) tuning control signal(s).

In some embodiments, the adjustment control signal represents or comprises a frequency offset value (the value may e.g. be positive or negative) to apply to offset the frequency of the feedback signal, i.e. in the feedback loop. Alternatively, or in addition, the frequency offset value may be derived on the basis of the adjustment control signal. The adjustment control signal may e.g. further comprise one or more channel settings, at least one modulation function/data, etc.

The frequency offset values or the adjustment control signal values (e.g. also compensating for temperature dependency) for a particular a crystal-free oscillator circuit for channel-based high-frequency radio communication as disclosed herein may e.g. be derived during calibration in the following way. During calibration temperature is set to different values (if also compensating for temperature dependency). Then a tuning (e.g. coarse tuning and/or fine tuning) of the crystal-free oscillator element and/or an adjustment of the adjustable frequency offset circuit via respective control signals (including the adjustment control signal) is determined e.g. by iterative approximation (such as binary search) so that the frequency of the generated high-frequency output signal (generated by the PLL circuit) matches a target value (e.g. within certain tolerances). The matching determined values of such control signals (coarse tuning and/or fine tuning of the crystal-free oscillator element and/or offset adjustment signal or frequency offset value) are then stored in data structure such as a table or similar in a memory. During operation, temperature is measured. This temperature is used to look up in the data structure, the appropriate value(s) of the aforementioned control signal(s). Alternatively, fewer temperature points can be used during calibration where a determined curve-fit interpolation or similar is used for a particular temperature to determine the control signal(s). This enables reduction of time and cost of calibration. These approaches may e.g. be expanded to repeat measurements with different target frequencies, resulting in a two-dimensional function in which measured temperature and the target frequency are the inputs and the required control signals are the output (in this way simplifying the necessary calculations during normal operation).

In some embodiments, the adjustable frequency offset circuit comprises a direct digital synthesizer (DDS) element or circuit (also sometimes referred to as a numerical control oscillator (NCO)) or similar controlling the frequency offset (e.g. in addition to enable modulation). A DDS is a type of frequency synthesizer that generally can create arbitrary waveforms.

In some embodiments, the adjustment control signal is provided in response to
an obtained or received temperature signal, representing a current operating temperature of at least a part of the crystal-free oscillator circuit, and
a predetermined relationship or function between operating temperatures of the at least a part of the crystal-free oscillator circuit and predetermined respective associated offset frequency values.

The frequency offset value may be derived on the basis of the obtained or received temperature signal and/or the predetermined relationship or function between operating temperatures of at least a part of the crystal-free oscillator circuit and predetermined respective associated frequency values. The adjustment control signal may be provided to the adjustable frequency offset circuit externally or alternatively the adjustable frequency offset circuit may be more advanced (e.g. comprising a processor, potentially memory, modulation functions, etc.) configured to generate the adjustment control signal (in such cases then e.g. receiving the temperature signal).

In some embodiments, the predetermined relationship or function has been determined for the particular crystal-free oscillator circuit, i.e. it is unique or at least specific for the crystal-free oscillator circuit in question.

In some embodiments, the crystal-free oscillator circuit is a solid-state integrated circuit or a part thereof.

In some embodiments, the temperature signal is provided by a (e.g. integrated circuit) temperature sensor circuit or element (e.g. comprised by the crystal-free oscillator circuit) configured to measure a current temperature of at least a part of the (e.g. integrated circuit) crystal-free oscillator circuit and supply the temperature signal in response thereto.

In some embodiments, the crystal-free oscillator circuit is a solid-state integrated circuit or a part thereof that further comprises a controllable (e.g. integrated circuit) heating element and a (e.g. integrated circuit) frequency counter (e.g. measuring/counting in reference to a known external accurate frequency), wherein the solid-state integrated circuit is configured to determine the predetermined relationship or function between operating temperatures and respective associated frequency values for a particular crystal-free oscillator circuit by incrementally or continuously increasing (for a time) a temperature of at least a part of the crystal-free oscillator circuit using the heating element and obtaining a number of temperature values and associated frequency values, obtained by a frequency counter at respective temperature values (i.e. the counted frequencies are obtained at respective temperatures). Alternatively, a number of temperature values and associated frequency offset values are obtained, where the associated frequency offset values is derived by taking the difference (at each temperature point) between the associated frequency values, as obtained by the frequency counter at respective temperature values, and predetermined target frequency values. The determined frequency values (or frequency offset values) and temperature values may e.g. be stored in a suitable memory and/or storage circuit. The associated frequency values may e.g. be counted for the frequency of the feedback signal, the frequency of the high-frequency reference signal, or the frequency of the high-frequency output signal. As an alternative, a cooling element could be used to decrease the temperature instead of increasing a temperature and using a heating element.

In some embodiments, the controllable heating element is a resistor circuit or element generating heat in response to being provided with an electrical current. In other embodiments, the controllable heating element is a so-called ‘hot plate’ (or as a further alternative a ‘cold plate’) to place a wafer or integrated circuit comprising the crystal-free oscillator circuit, or more typically a large number of crystal-free oscillator circuits, on e.g. during post-manufacturing calibration.

In some embodiments, a number of voltage values for a supply voltage for the crystal-free oscillator circuit may be obtained and stored during initial calibration for each temperature value as well (in addition to the frequency values). This enables ongoing compensation for a varying supply voltage e.g. due to ‘aging’, a less reliable battery power source, etc.

In some embodiments, the crystal-free oscillator circuit further comprises a second static frequency divider being located in the feedback loop and being configured to divide down a frequency of the feedback signal by a factor being a second predetermined positive integer (M), and wherein the adjustable frequency offset circuit is configured to offset the frequency of the feedback signal after being divided down by the second frequency divider (and at least in some embodiments before being divided down by the first frequency divider for embodiments comprising such a first frequency divider). It is noted, that a second frequency divider does not necessarily require a first frequency divider to be present although it often will be, at least in some embodiments. By dividing down the frequency before the adjustable frequency offset circuit offsets the frequency of the feedback signal enables that the adjustable frequency offset circuit will operate at a lower frequency (being the divided down frequency) whereby power consumption is reduced.

In some embodiments, the crystal-free oscillator circuit further comprises a third static frequency divider being located between the crystal-free oscillator element and the PLL circuit and being configured to divide down a frequency of an output signal of the crystal-free oscillator element by a factor being a third predetermined positive integer (R) to generate the high-frequency reference signal. Such a divider may improve flexibility in relation to the frequency output of the PLL.

In some embodiments, the frequency generator and the second static frequency divider each provide a first and a second output, and the adjustable frequency offset circuit comprises a first mixer or modulator, a second mixer or modulator, and an adding element, wherein the adjustable frequency offset circuit is configured
to mix or modulate, by the first mixer or modulator, the first output from the frequency generator and the first output of the second frequency divider resulting in a first mixed or modulated signal,
to mix or modulate, by the second mixer or modulator, the second output from the frequency generator and the second output of the second frequency divider resulting in a second mixed or modulated signal, and
to add, by the adding element, the first and the second mixed or modulated signals and supply the resulting signal as output of the adjustable frequency offset circuit.

This efficiently enables rejecting of a mirror product. If, e.g. each of the first and the second output of the frequency generator and the second static frequency divider respectively provides outputs comprising Q (of the quadrature signal) and I (of the in-phase signal) quadrature is provided thereby rejecting of a mirror product.

In some embodiments, the first predetermined positive integer (N) is 2 (or about 2) and/or the second predetermined positive integer (M) is 2 or 4 (or about 2 or about 4). For some embodiments comprising a first, second, and a third divider, N may be 2, M may be 4, and R may be 4, but actual values may depend on specific implementation or use. By not dividing down too much, it is ensured that phase noise generated by the division is minimized or at least does not become too large for channel-based high-frequency radio communication uses. It is noted, that is some embodiments depending on use and implementation, one or more of the first (N), the second (M), and/or the third frequency (R) is not used.

In some embodiments, the crystal-free oscillator circuit further comprises
a phase frequency detector (PFD) being configured to receive the high-frequency reference signal and the feedback signal and to derive at least one phase error signal in response thereto, and
a low-pass filter (LPF) being configured to low-pass filter the at least one phase error signal and to derive an oscillator input signal in response thereto, wherein the crystal-free oscillator element is configured to derive the high-frequency output signal in response to the oscillator input signal.

In some embodiments, the high-frequency reference signal has a frequency of about 2 GHz, or of about 2 GHz or more. Such a relatively high operating frequency is advantageous as it reduces or minimizes phase noise and improves the q-factor even further (compared to a crystal-free oscillator element operating at lower frequency). In some embodiments and depending on implementation and/or use, the operating frequency of the crystal-free oscillator element may be lower that about 2 GHz or lower than about 1 GHz depending on the performance (in relation to phase noise) of the crystal-free oscillator element or circuit; the higher phase noise generation, the higher operating frequency should be used.

In at least some embodiments, the crystal-free oscillator circuit is implemented as a monolithic integrated circuit.

In some embodiments, the output signal is provided to a channel-based radio communication element or system, e.g. a Bluetooth, Bluetooth Low Energy or other eligible communication element or system.

According to an additional aspect is provided a method of deriving a unique temperature and frequency profile for a particular crystal-free oscillator circuit (i.e. the temperature and frequency profile is unique and specific for the particular crystal-free oscillator circuit), e.g. as disclosed herein, the method comprising:
determining a relationship or function between operating temperatures and respective associated frequency values of a feedback signal or a reference signal or a high-frequency output signal of the particular crystal-free oscillator circuit of the particular crystal-free oscillator circuit by incrementally or continuously increasing (or alternatively decreasing) a temperature of at least a part of the particular crystal-free oscillator circuit using a heating element (or alternatively a cooling element) and obtaining, and storing in a memory and/or storage, a number of temperature values and associated frequency values, obtained by a frequency counter at respective temperature values or, alternatively, a number of temperature values and associated frequency offset values derived from frequency target values and associated frequency values, obtained by the frequency counter at respective temperature values.

In some embodiments, the method steps above are repeated for a number of different particular crystal-free oscillator circuits being part of a same wafer or similar. It is noted, that in general, the unique temperature and frequency profiles for different crystal-free oscillator circuits will vary even when the crystal-free oscillator circuits have been manufactured as part of a same single wafer or similar.

Alternatively, a cooling element (or a combined heating/cooling element) could be used to decrease the temperature instead of increasing a temperature and using a heating element.

According to a further aspect is provided a channel-based radio communication device or system comprising a crystal-free oscillator circuit as disclosed herein. In some embodiments, the channel-based radio communication device or system is a (one time use or few time use) disposable and/or a time-limited (within a certain predetermined period of time) use product.

According to another aspect is provided a medical device comprising a crystal-free oscillator circuit as disclosed herein or a channel-based radio communication device or system as disclosed herein.

In some embodiments, the medical device is a liquid drug delivery device, e.g. an injection device for delivering set doses of a liquid drug, comprising
a housing storing, in use, a cartridge (or other container) having a distal end being closed by a septum or similar and a proximal end being closed by a movable plunger or similar defining an interior containing the liquid drug, and
a needle cannula having a distal end with a tip and a proximal end, which proximal end is in liquid communication with the interior of the cartridge when the needle cannula and the cartridge is mounted in the liquid drug delivery device.

In some embodiments, the liquid drug delivery device is an injection device for delivering set doses of a liquid drug. In some embodiments, the liquid drug delivery device is an insulin delivery device. In some embodiments, the liquid drug delivery device is a pen-based injection device.

In some embodiments, the medical device is a (one time use or few time use) disposable and/or a time-limited (within a certain predetermined period of time) use product.

In some embodiments, the injection device is an insulin injection device or a disposable and/or time-limited use insulin injection device.

The medical device/the liquid drug delivery device may e.g. be automatic, semi-automatic, or manual.

A crystal-free oscillator circuit for channel-based radio communication as disclosed herein is particularly advantageous for use in or with such disposable and/or a time-limited devices as the costs for a communications capable device is reduced significantly by avoiding the use of a crystal-based oscillator.

In this way, it is possible to provide communications related functionality (sending/receiving information, data, etc.) even for more or less disposable and/or a time-limited products e.g. involving only a single use, a few uses, or uses only for a limited amount of time such as for about a month or couple of weeks or less.

These embodiments and/or aspects (including the main embodiments and/or aspects) provide advantages in connection with radio based communications systems using channel synthesis or similar.

In some embodiments, the crystal-free oscillator circuit as disclosed herein and embodiments thereof is for channel-based radio communication according to the Bluetooth standard or alternatively for the Bluetooth Low Energy (BLE) standard.

In other embodiments, the crystal-free oscillator circuit as disclosed herein and embodiments thereof is for channel-based radio communication according to other standards including one or more selected from the group of standards according to 3G, 4G, and/or 5G broadband cellular networks, wifi, near field communication (NFC) networks, gigabit networks, wireless local area networks (WLAN), global system for mobile communications (GSM) networks, (wireless) code division multiple access ((W)CDMA) networks, narrowband radio communications systems, universal mobile telecommunications system (UMTS), or in general any other wireless radio-based communications network having relatively high phase error requirements.

According at least to some aspects/embodiments, the crystal-free oscillator circuit as disclosed herein specifically does not comprise a MEMS (Micro-Electro-Mechanical Systems) oscillator and not any other mechanically based oscillator, i.e. these are disclaimed.

According at least to some aspects/embodiments, the crystal-free oscillator circuit as disclosed herein does not comprise a fractional divider or fractional-N divider, i.e. these are disclaimed (at least according to some aspects/embodiments also in combination with the above disclaimer of mechanically based oscillators).

### Definitions

An “injection pen” is typically an injection apparatus having an oblong or elongated shape somewhat like a fountain pen for writing. Although such pens usually have a tubular cross-section, they could easily have a different cross-section such as triangular, rectangular or square or any variation around these geometries.

As used herein, the term “drug” is meant to encompass any drug-containing flowable medicine capable of being passed through a delivery means such as a hollow needle in a controlled manner, such as a liquid, solution, gel or fine suspension. Representative drugs includes pharmaceuticals such as peptides, proteins (e.g. insulin, insulin analogues and C-peptide), and hormones, biologically derived or active agents, hormonal and gene based agents, nutritional formulas and other substances in both solid (dispensed) or liquid form.

The term “needle cannula” is used to describe the actual conduit performing the penetration of the skin during injection. A needle cannula is usually made from a metallic material such as e.g. stainless steel and connected to a hub to form a complete injection needle also often referred to as a “needle assembly”. A needle cannula could however also be made from a polymeric material or a glass material. The hub also carries the connecting element(s) for connecting the needle assembly to an injection apparatus and is usually moulded from a suitable thermoplastic material. The “connection element(s)” could as examples be a luer coupling, a bayonet coupling, a threaded connection or any combination thereof—e.g. a combination as described in EP 1,536,854.

“Cartridge” is the term used to describe the container containing the drug. Cartridges are usually made from glass but could also be moulded from any suitable polymer. A cartridge or ampoule is preferably sealed at one end by a pierceable membrane referred to as the “septum” which can be pierced e.g. by the non-patient end of a needle cannula. The opposite end is typically closed by a plunger or piston made from rubber or a suitable polymer. The plunger or piston can be slidable moved inside the cartridge. The space between the pierceable membrane and the movable plunger holds the drug which is pressed out as the plunger decreased the volume of the space holding the drug. However, any kind of container—rigid or flexible—can be used to contain the drug.

Using the term “automatic” in conjunction with injection device means that, the injection device is able to perform the injection automatically without the user of the injection device delivering the force needed to expel the drug. The force is typically delivered by an electric motor or by a spring as herein described. The spring is usually strained by the user during dose setting. However, such springs are usually pre-strained in order to avoid problems of delivering very small doses. Alternatively, the spring can be fully preloaded by the manufacturer with a preload sufficient to empty the drug cartridge through a number of doses. Typically, the user activates a latch mechanism e.g. in the shape of a button on the injection device to release the force accumulated in the spring when carrying out the injection. The release mechanism can also be coupled to a proximally located injection button.

All references, including publications, patent applications, and patents, cited herein are incorporated by reference in their entirety and to the same extent as if each reference were individually and specifically indicated to be incorporated by reference and were set forth in its entirety herein.

All headings and sub-headings are used herein for convenience only and should not be constructed as limiting the invention in any way.

The use of any and all examples, or exemplary language (e.g. such as) provided herein, is intended merely to better illuminate the invention and does not pose a limitation on the scope of the invention unless otherwise claimed. No language in the specification should be construed as indicating any non-claimed element as essential to the practice of the invention. The citation and incorporation of patent documents herein is done for convenience only and does not reflect any view of the validity, patentability, and/or enforceability of such patent documents.

This invention includes all modifications and equivalents of the subject matter recited in the claims appended hereto as permitted by applicable law.

### BRIEF DESCRIPTION OF THE DRAWINGS

FIG. 1 schematically illustrates one exemplary embodiment of crystal-free oscillator circuit for channel-based high-frequency radio communication;

FIG. 2 schematically illustrates another exemplary embodiment of crystal-free oscillator circuit for channel-based high-frequency radio communication;

FIG. 3 schematically illustrates yet another exemplary embodiment of crystal-free oscillator circuit for channel-based high-frequency radio communication;

FIG. 4 schematically illustrates another exemplary embodiment of crystal-free oscillator circuit for channel-based high-frequency radio communication;

FIG. 5 schematically illustrates a further exemplary embodiment of crystal-free oscillator circuit for channel-based high-frequency radio communication;

FIG. 6 schematically illustrates an integrated circuit comprising an embodiment of a crystal-free oscillator circuit for channel-based high-frequency radio communication as disclosed herein together with additional elements;

FIG. 7 schematically illustrates one embodiment of a method of generating pairs of temperature and frequency values for a specific crystal-free oscillator circuit;

FIG. 8 schematically illustrates a device, and in particular a liquid drug delivery device, comprising a crystal-free oscillator circuit for channel-based high-frequency radio communication as disclosed herein;

FIG. 9 schematically illustrates an exemplary embodiment of a crystal-free oscillator element according to various embodiments; and

FIG. 10 schematically illustrates further details of the crystal-free oscillator element of FIG. 9 together with additional elements.

### DETAILED DESCRIPTION

Various aspects and embodiments of a crystal-free oscillator circuit for channel-based high-frequency radio communication, a channel-based high-frequency radio communication device or system, a medical device comprising a crystal-free oscillator circuit for channel-based high-frequency radio communication, and a method of deriving a unique temperature and frequency adjustment profile or similar for a particular crystal-free oscillator circuit, as disclosed herein will now be described with reference to the figures.

When/if relative expressions such as “upper” and “lower”, “right” and “left”, “horizontal” and “vertical”, “clockwise” and “counter clockwise” or similar are used in the following terms, these only refer to the appended figures and not to an actual situation of use. The shown figures are schematic representations for which reason the configuration of the different structures as well as their relative dimensions are intended to serve illustrative purposes only.

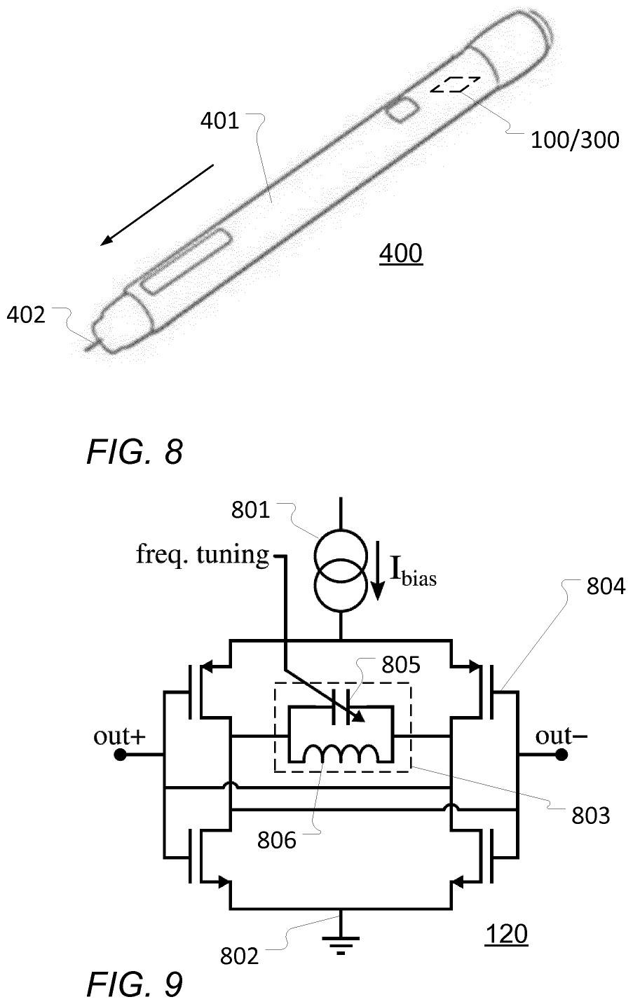

In the context of the medical device, it may be convenient to define that the term “distal end” in the relevant appended figure is meant to refer to the end of the medical device which usually carries an injection needle and as depicted e.g. in FIG. 8 whereas the term “proximal end” is meant to refer to the opposite end pointing away from the injection needle.

Some of the different components are only disclosed in relation to a single embodiment of the invention, but is meant to be included in the other embodiments without further explanation.

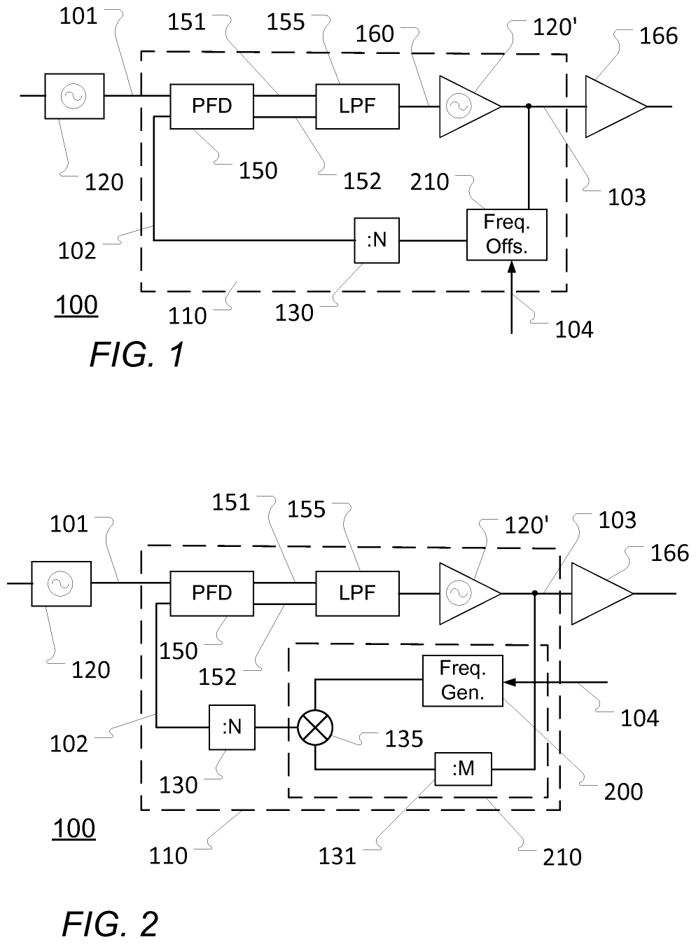

FIG. 1 schematically illustrates one exemplary embodiment of crystal-free oscillator for channel-based high-frequency radio communication.

Illustrated is an exemplary embodiment of a crystal-free oscillator circuit 100 for channel-based high-frequency radio communication as disclosed herein, where the crystal-free oscillator circuit 100 comprises a crystal-free oscillator element 120 configured to provide a high-frequency reference signal (where the high-frequency reference signal has a frequency of at least about 1 GHz or at least about 2 GHz) and a phase-locked loop (PLL) circuit 110 having a feedback loop and comprising a PLL oscillator 120′ as disclosed herein. The PLL circuit 110 is configured to receive the high-frequency reference signal 101 and to provide or generate a feedback signal 102 in/to the feedback loop. It is noted that the feedback signal 102 is to designate the signal in the whole feedback loop even though, the feedback signal will be modified, processed, changed, etc. by various elements as disclosed herein. The PLL circuit 110 is furthermore configured to provide or generate a high-frequency output signal 103 in response to the high-frequency reference signal 101 and the feedback signal 102 where the feedback signal 102 is dependent on an earlier instance of the output signal 103. The crystal-free oscillator element 120 is, at least in some embodiments, an LC-based oscillator.

In some embodiments, and as shown, the crystal-free oscillator circuit 100 further comprises a phase frequency detector (PFD) 150 receiving the high-frequency reference signal 101 and the feedback signal 102, where the PFD 150 is configured to derive at least one phase error signal 151, 152 in response to the received reference and feedback signals 101, 102. The crystal-free oscillator circuit 100 additionally comprises a low-pass filter (LPF) 155 configured to low-pass filter the at least one phase error signal 151, 152 and to derive an oscillator input signal 160 in response thereto, where the oscillator input signal 160 is provided to the PLL oscillator 120′ to derive or generate the output signal 103, which is the output of the PLL circuit 110 and is also used in the feedback loop.

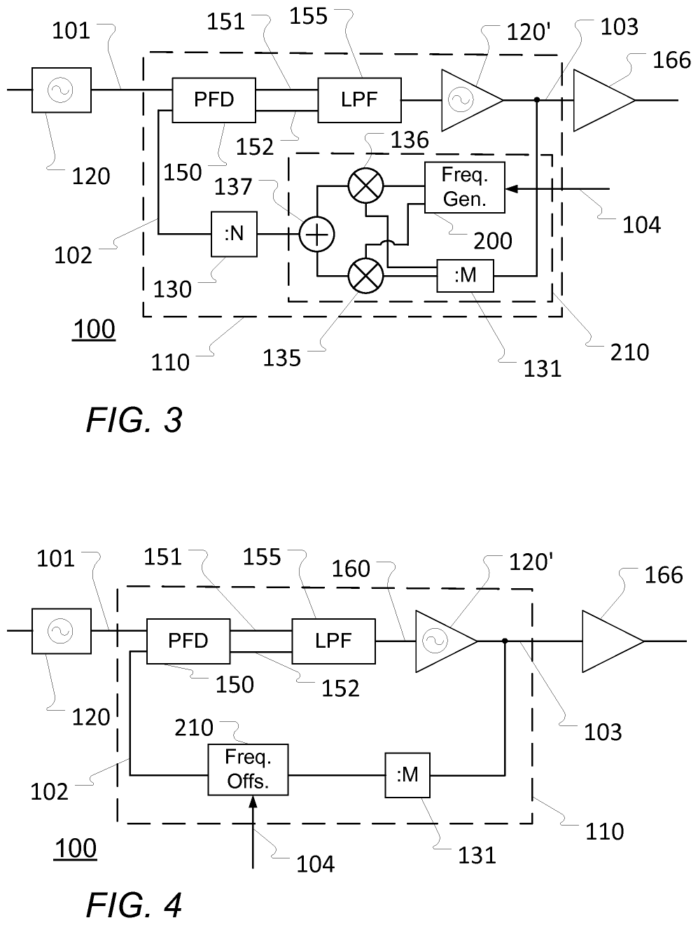

As disclosed herein, the crystal-free oscillator circuit 100 further comprises (in this and corresponding embodiments) an adjustable frequency offset circuit 210, as disclosed herein, comprising a frequency generator (not shown see e.g. 200 in FIGS. 2 and 3), where the adjustable frequency offset circuit 210 is configured to offset a frequency of the feedback signal 102 in response to an adjustment control signal 104, wherein the adjustable frequency offset circuit 210 is located in the feedback loop. The frequency generator is configured to generate a periodic signal having a frequency set under the control of the adjustable frequency offset circuit 210 and e.g. in particular set in dependency of the adjustment control signal 104. The precise location of the adjustable frequency offset circuit 210 may vary according to various embodiments (see e.g. FIGS. 2-4 for other exemplary locations/layouts). What is significant is that the adjustable frequency offset circuit 210 causes, as disclosed herein, an offset of the frequency of the feedback signal 102 before ultimately being used by the PLL oscillator element 120′. The adjustment control signal 104 may be external (to the adjustable frequency offset circuit 210 as shown) or be generated internally (then requiring a more advanced adjustable frequency offset circuit 210). The crystal-free oscillator circuit 100 is additionally configured to compensate for a temperature dependency of the crystal-free oscillator circuit 100 where the compensation is done in response to a measured current operating temperature. This temperature dependency compensation is in some embodiments done by adjusting signals or other aspects of the crystal-free oscillator element 120 e.g. as disclosed herein. Alternatively, the temperature dependency compensation is done by the adjustable frequency offset circuit 210, whereby the needed temperature compensation data is or may be included as part of the adjustment control signal 104.

At least in some embodiments, the adjustment control signal 104 represents or comprises a frequency offset value (may be both positive and negative) used to offset the frequency of the feedback signal 102 when applied. The adjustment control signal 104 may in addition represent or comprise communication channel parameters and/or a modulation function or data. The adjustment control signal 104 may in some other embodiments be different as disclosed herein.

In some embodiments, the frequency generator 200 comprises or is a direct digital synthesizer (DDS) element or circuit (also sometimes referred to as a numerical control oscillator (NCO)) or similar providing the frequency to offset the feedback signal. A DDS is a type of frequency synthesizer that generally can create periodical functions with arbitrary frequencies.

In some embodiments, and as shown, the crystal-free oscillator circuit 100 comprises a first static frequency divider 130 configured to divide down a frequency of the feedback signal 102 by a factor being a first predetermined positive integer N. N may e.g. be 2 or 4 or any other suitable integer according to a specific implementation (e.g. taking power consumption into account) of the crystal-free oscillator circuit 100. By not dividing down by (too) large integers it is ensured that the phase noise is not increased (too much). It is also relatively simple to obtain appropriate quadrature signals from a 2 or 4 divider.

In the shown embodiment, the first frequency divider 130 is located in the feedback loop after the adjustable frequency offset circuit 210 (or at least after the adjustable frequency offset circuit 210 has offset the frequency of the feedback signal 102 as disclosed herein). Alternatively (see e.g. FIG. 4), the first frequency divider 130 (then designated a second frequency divider (M) 131) is located in the feedback loop before the adjustable frequency offset circuit 210 (or at least before the adjustable frequency offset circuit 210 has offset the frequency of the feedback signal 102 as disclosed herein).

The resulting frequency offset feedback signal 102 is then divided down by the first frequency divider 130 (if present) and the resulting signal is provided as input to the PFD 150 together with the high-frequency reference signal 101.

In the shown and corresponding embodiments, the frequencies of the crystal-free oscillator circuit 100/the PLL circuit 110 may be seen as being governed according to:

$${f}_{ref} = \frac{1}{N} \cdot ( {f}_{v c o} \pm {f}_{DDS} ) ⇒ {f}_{0} = {f}_{vco} = N \cdot ( {f}_{ref} ∓ {f}_{DDS} )$$

where f_{ref} is the frequency of the high-frequency reference signal 101, f_{vco} is the frequency of the PLL oscillator 120, f₀ is the frequency of the high-frequency output signal 103, f_{DDS} is the frequency offset value offsetting the frequency of the input of the PLL circuit (by the adjustable frequency offset circuit 210, e.g. comprising a frequency generator such as a DDS, offsetting the feedback signal as disclosed herein), and N is the integer value of the first frequency divider 130, each at each time instance.

In the shown and corresponding embodiments, the high-frequency output signal 103 is provided to an amplifier 166 e.g. acting as a power amplifier for an antenna of a channel-based high-frequency radio communication system or device.

In some embodiments, the crystal-free oscillator circuit 100 further comprises a second (static) frequency divider being located in the feedback loop and being configured to divide down the frequency of the feedback signal 102 by a factor being a second predetermined positive integer M (see e.g. 131 in FIGS. 2 and 3) and/or a third frequency divider being located between the crystal-free oscillator element and the PLL circuit and being configured to divide down a frequency of an output signal of the crystal-free oscillator element by a factor being a third predetermined positive integer (R) to generate the high-frequency reference signal (in addition, at least in some embodiments, to a first frequency divider 130 dividing down by N).

FIG. 2 schematically illustrates another exemplary embodiment of crystal-free oscillator for channel-based high-frequency radio communication.

Illustrated is an exemplary embodiment of a crystal-free oscillator circuit 100 for channel-based high-frequency radio communication as disclosed herein, where the crystal-free oscillator circuit 100 corresponds to the one shown and explained in connection with FIG. 1 except as noted in the following.

In this and corresponding embodiments, the crystal-free oscillator circuit 100 comprises a second static frequency divider 131 (in addition, at least in some embodiments, to a first frequency divider 130 dividing down by N or in some alternative embodiments instead) being located in the feedback loop and being configured to divide down the frequency of the feedback signal 102 by a factor being a second predetermined positive integer M. The adjustable frequency offset circuit 210 is configured to offset the frequency of the feedback signal 102 after being divided down M times by the second frequency divider 131 and before being divided down by first frequency divider 130 (for embodiments comprising such a first frequency divider). Dividing down the frequency before offsetting the frequency of the feedback signal reduces power consumption since the adjustable frequency offset circuit accordingly operates at a lower frequency (being the divided down frequency). M may e.g. be 2 or 4 or any other suitable integer according to a specific implementation of the crystal-free oscillator circuit 100. For embodiments also comprising a first frequency divider, both M and N may each e.g. be 2. Alternatively, N may be 2 and M may be 4. In the shown and corresponding embodiments, the adjustable frequency offset circuit 210 comprises the second frequency divider 131.

More specifically, the frequency generator 200 (of the adjustable frequency offset circuit 210) signal is mixed or modulated with the feedback signal 102 (after being divided down M times) by mixer or modulator 135 thereby offsetting the frequency of the feedback signal 102 as disclosed herein and e.g. carrying out further processing. It is noted that the feedback signal 102 as received by the adjustable frequency offset circuit 210, or more specifically the second frequency divider (M) 131, corresponds to the high-frequency output signal 103 generated by the PLL oscillator 120′.

The resulting frequency offset feedback signal 102 (here being a processed signal based on the high-frequency output signal 103) is then divided down by the first frequency divider 130 (if present) and the resulting signal is provided as input to the PFD 150 together with the high-frequency reference signal 101.

In the shown and corresponding embodiments (using only a first and a second frequency divider), the frequencies of the crystal-free oscillator circuit 100/the PLL circuit 110 may be seen as being governed according to:

$${f}_{ref} = \frac{1}{N} \cdot ( \frac{{f}_{v c o}}{M} \pm {f}_{DDS} ) ⇒ {f}_{0} = {f}_{v c o} = M \cdot ( N \cdot {f}_{ref} ∓ {f}_{DDS} ) ⇒ {f}_{DDS} = \frac{{f}_{0}}{M} - N \cdot {f}_{ref}$$

where f_{ref} is the frequency of the high-frequency reference signal 101, f_{vco} is the frequency of the PLL oscillator 120, f₀ is the frequency of the high-frequency output signal 103, f_{DDS} is the frequency offset value offsetting the frequency of the input of the PLL circuit (by the adjustable frequency offset circuit 210, e.g. comprising a DDS, offsetting the feedback signal), N is the integer value of the first frequency divider 130, and M is the integer value of the second frequency divider 131, each at each time instance.

As one example, in case of Bluetooth Low Energy (BLE) communication, the channel spacing is 2 MHz, f₀ (the carrier) is in the range from (about) 2400 MHz to 2480 MHz. If the f_{vco} is about 2000 MHz, this frequency is divided by 4 resulting in a f_{ref} of about 500 MHz.

If the first and second frequency dividers 130, 131 each divides by 2, then the frequency range of f_{DDS} can be calculated according to:

$${f}_{DDS} = \frac{{f}_{0}^{′} + Δ f}{M} - N \cdot {f}_{ref} ⇒ {f}_{DDS} = \frac{2000 + Δ f}{2} - 2 \cdot 400 = 200 + \frac{Δ f}{2}$$

where the frequency Δf is in the range from 0 MHz to 80 MHz and Δf may be expressed as:

Δf=n·f_{chl}+f_{T}+f_{M}(t)

where n is a channel scaler or channel (0 to 40 according to BLE), f_{chl} is channel spacing (being 2 MHz according to BLE), f_{T} is the offset and temperature compensated frequency, and f_{M}(t) is a modulation frequency function versus time.

Other embodiments may correspond to the one shown in FIG. 2 but without the first frequency divider 130.

FIG. 3 schematically illustrates yet another exemplary embodiment of crystal-free oscillator for channel-based high-frequency radio communication.

Illustrated is an exemplary embodiment of a crystal-free oscillator circuit 100 for channel-based high-frequency radio communication as disclosed herein, where the crystal-free oscillator circuit 100 corresponds to the one shown and explained in connection with FIG. 2 except as noted in the following.

Instead of providing one output from each of the frequency generator 200 and the second frequency divider 131 as in FIG. 2, the frequency generator 200 and the second frequency divider 131 each provide two outputs (respectively comprising Q (of the quadrature signal) and I (of the in-phase signal)), where a first output from the frequency generator 200 and a first output of the second frequency divider 131 is mixed or modulated by mixer or modulator 135 (as described in connection with FIG. 2) (resulting in a first mixed or modulated signal) and a second output from the frequency generator 200 and a second output of the second frequency divider 131 is mixed or modulated by an additional mixer or modulator 136 (as described in connection with FIG. 2) (resulting in a second mixed or modulated signal). The two resulting signals are then added by adding element 137 and the result of the addition is output by the adjustable frequency offset circuit 210, where the resulting frequency offset feedback signal 102 (here being a processed signal based on the high-frequency output signal 103) then is divided down by the first frequency divider 130 N times (if present) where the potentially N-divided down frequency offset feedback signal 102 is provided as input to the PFD 150 together with the reference signal 101. This arrangement provides quadrature of the outputs of the frequency generator 200 and the second frequency divider 131 enabling an efficient rejection of a mirror product. Alternatively, a filter (with a relatively low order) may be used to enable rejection of mirror a product (and may e.g. be used in embodiments corresponding to the ones of FIGS. 1 and 2 and others). However, such a filter is fairly complex to realise in a usable manner in an integrated circuit.

Other embodiments may correspond to the one shown in FIG. 3 but without the first frequency divider 130. As mentioned, the crystal-free oscillator 100 may in some embodiments comprise a third static frequency divider being located between the crystal-free oscillator element and the PLL circuit and being configured to divide down a frequency of an output signal of the crystal-free oscillator element by a factor being a third predetermined positive integer (R) to generate the high-frequency reference signal

The quadrature arrangement may also be used for other embodiments, e.g. the ones (and corresponding ones) shown in FIGS. 1, 4 and 5.

FIG. 4 schematically illustrates another exemplary embodiment of crystal-free oscillator for channel-based high-frequency radio communication.

Illustrated is an exemplary embodiment of a crystal-free oscillator circuit 100 for channel-based high-frequency radio communication as disclosed herein, where the crystal-free oscillator circuit 100 corresponds to the one shown and explained in connection with FIG. 1 except as noted in the following. In FIG. 4, a second (static) frequency divider 131, dividing down by M) is located in the feedback loop before the adjustable frequency offset circuit 210 (or at least before the adjustable frequency offset circuit 210 offsets the frequency of the feedback signal 102 as disclosed herein) rather than being located after the adjustable frequency offset circuit 210 or after the adjustable frequency offset circuit 210 has offset the frequency of the feedback signal 102 as in FIG. 1 (where such a frequency divider is designated a first frequency divider (N)).

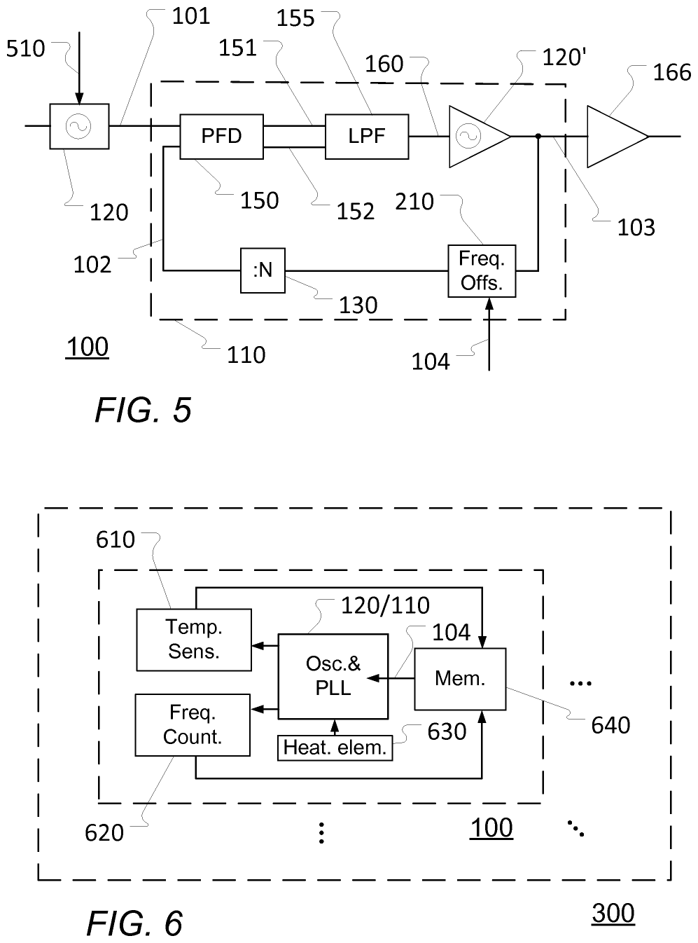

FIG. 5 schematically illustrates a further exemplary embodiment of crystal-free oscillator for channel-based high-frequency radio communication.

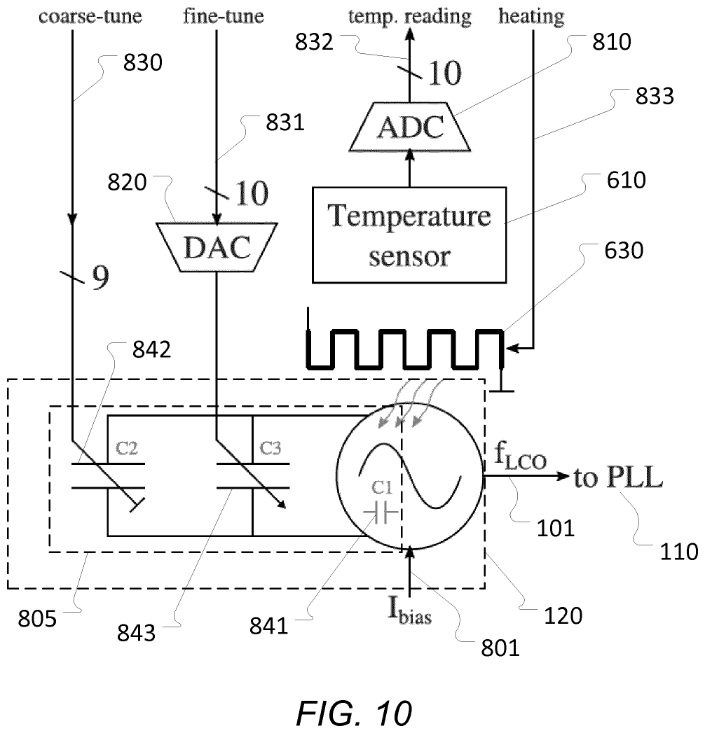

Illustrated is an exemplary embodiment of a crystal-free oscillator circuit 100 for channel-based high-frequency radio communication as disclosed herein, where the crystal-free oscillator circuit 100 corresponds to the one shown and explained in connection with FIG. 1 except as noted in the following. In FIG. 5, instead of compensating for a temperature dependency of the crystal-free oscillator circuit via an adjustable frequency offset circuit 210 as disclosed herein (i.e. in addition to offsetting the frequency of the feedback signal 102), then the temperature dependency compensation is done by adjusting signals or other aspects of the crystal-free oscillator element 120. More particularly, a temperature compensation signal 510 is received or used by the crystal-free oscillator element 120. In some embodiments where the crystal-free oscillator element 120 is an LC-based oscillator, this may e.g. be done by controlling a capacitor value of the LC oscillator e.g. as described in connection with FIGS. 9 and/or 10.

FIG. 6 schematically illustrates an integrated circuit comprising an embodiment of a crystal-free oscillator for channel-based high-frequency radio communication as disclosed herein together with additional elements.

Illustrated is a crystal-free oscillator circuit 100 for channel-based high-frequency radio communication as disclosed herein comprising a crystal-free oscillator element 120 and a PLL 110, e.g. a crystal-free oscillator element 120 and a PLL 110 as shown and/or explained in connection with any one of FIGS. 1-5. In this particular exemplary and corresponding embodiments, the crystal-free oscillator circuit 100 further comprises a temperature sensor 610, a frequency counter 620, a controllable heating element 630, and a memory and/or storage 640.

In some embodiments, all these elements may are manufactured as a solid-state monolithic integrated circuit. It is possible to manufacture a large number of such integrated circuits ‘on-chip’/‘on-silicium’ e.g. on a single wafer or similar 300. This is opposed e.g. to crystal-based oscillators that cannot completely be manufactured as a monolithic integrated circuit due to the resonator part of such comprising the crystal.

The controllable heating element 630 is configured to heat at least a part of the PLL 110 (e.g. a part comprising the adjustable frequency offset circuit 210) as indicated by the arrow pointing to the crystal-free oscillator element 120 and the PLL 110 from the heating element 630. In some embodiments, the controllable heating element 630 is a resistor circuit or element generating heat in response to being provided with an electrical current. In some alternative embodiments, the crystal-free oscillator circuit 100 or the PLL 110, comprises a controllable cooling element (or the heating element 630 is configured also to be able to cool) configured to cool at least a part of the crystal-free oscillator element 120 and/or the PLL 110. Cooling may e.g. be used to cool at least a part of the crystal-free oscillator element 120 and/or the PLL 110 to a predetermined starting temperature used during initial calibration as disclosed elsewhere herein. In other embodiments, the controllable heating element may be replaced by a so-called ‘hot plate’ (or as a further alternative a ‘cold plate’ instead or as an addition).

The temperature sensor 610 is configured to measure (as indicated by the arrow pointing from the crystal-free oscillator element 120 and the PLL 110 to the temperature sensor 610) the temperature of at least a part of the crystal-free oscillator element 120 and/or the PLL 110 or alternatively at least a part of the crystal-free oscillator circuit 100 resulting in a value representing a current operating temperature of the crystal-free oscillator element 120 and/or the PLL 110 (or the crystal-free oscillator circuit 100).

The frequency counter 620 is configured to measure or count (e.g. in reference to a known external accurate frequency) the frequency of the feedback signal 102, the frequency of the high-frequency reference signal 101, or the frequency of the high-frequency output signal 103 (e.g. for embodiments such as shown in FIGS. 1-5) during initial calibration. The frequency is—at least in some embodiments—measured or counted where the adjustable frequency offset circuit or the frequency generator (see e.g. 210 or 200 in FIGS. 1-5) is located or where the adjustable frequency offset circuit offsets the frequency as disclosed herein. Alternatively, the frequency may be counted (e.g. for embodiments such as shown in FIGS. 9 and 10) near the crystal-free oscillator element 120 or the LC-based oscillator (LCO) 120.

These elements enable efficient determination of the predetermined relationship or function (used according to at least some embodiments by the adjustable frequency offset circuit) between operating temperatures and respective associated counted or measured frequencies (used according to at least some embodiments by the adjustable frequency offset circuit to compensate for respective frequency deviation) for the particular crystal-free oscillator circuit 100. From the respective associated counted or measured frequencies and the known external accurate frequency, an offset frequency value (may be positive or negative) can be determined for the particular crystal-free oscillator circuit 100 at respective operating temperatures

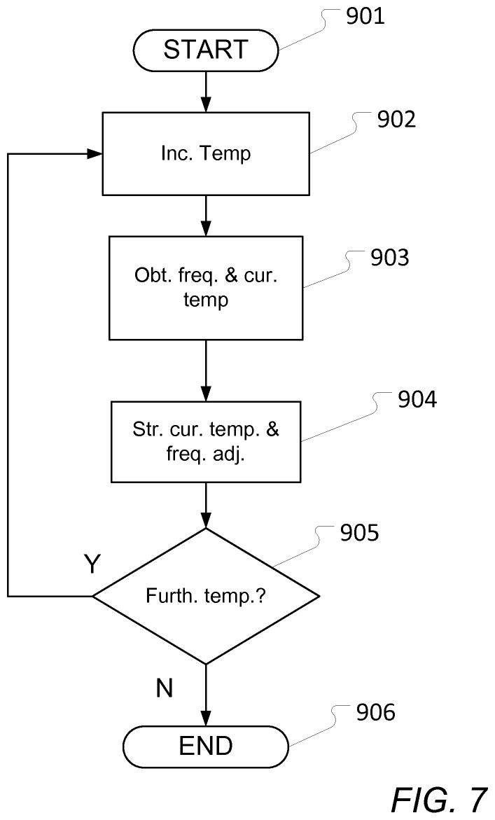

This may e.g. be done by incrementally or continuously increasing the temperature using the heating element 630 (or alternatively decrease using a cooling element) and for each temperature value then obtaining a frequency by the frequency counter 620. Each temperature value and obtained frequency value (or each temperature value and an frequency offset value derived by finding the difference between an obtained frequency value and a frequency target value) may then e.g. be stored in a suitable memory and/or storage 640 (as indicated by the arrows pointing to the memory and/or storage 640) as a data structure representing a profile, a table of pairs, or other suitable data structure of operating temperatures of the crystal-free oscillator element 120 and/or PLL 110 (or alternatively of the crystal-free oscillator circuit 100) and associated frequency values. Further details of such exemplary generation of the predetermined relationship or function is e.g. shown and given in connection with FIG. 7.

The stored values or profile may e.g. then be supplied during operation from the memory and/or storage 640 as indicated by arrow 104 or arrow 510 in FIG. 5 to the adjustable frequency offset circuit of the PLL or to the crystal-free oscillator circuit as disclosed herein. During operation, a current temperature may then be measured or obtained (by the temperature sensor 610) and from that and using the data of the memory and/or storage 640 it is possible to derive a frequency (and/or phase) offset value that is to be used by the adjustable frequency offset circuit when at (or near) the associated temperature to compensate for the frequency difference to a target frequency. It is noted, that the frequency values obtained and stored during initial calibration does not need to be absolute values but just need to correlate with the respective temperatures obtained during initial calibration. This is much simpler and reduces the needed complexity of the frequency counter 620 and also avoids the need of calibrating the temperature sensor 610. The current temperature obtained during operation does not need to exist as a temperature value in the memory and/or storage 640 as the associated frequency e.g. can be interpolated using the stored temperature and frequency values and the obtained temperature value.

FIG. 7 schematically illustrates one embodiment of a method of generating pairs of temperature and frequency values for a specific crystal-free oscillator circuit.

Illustrated is a flow-chart of one embodiment of a method of generating pairs of temperature and frequency values for a specific crystal-free oscillator circuit as disclosed herein e.g. during initial (post-manufacture) calibration.

At step 901, the method starts and potentially is initialized, etc. This may e.g. involve setting a starting temperature (e.g. by cooling) of a particular crystal-free oscillator circuit, e.g. of the crystal-free oscillator element 120 and/or the PLL (see e.g. 100, 120, and 110 in FIGS. 1-6).

At step 902, the temperature of (at least a part of) the particular crystal-free oscillator circuit, e.g. the crystal-free oscillator element 120 and/or the PLL is increased incrementally or continuously.

At step 903, a current temperature of the crystal-free oscillator circuit, e.g. the crystal-free oscillator element 120 and/or the PLL and a measured frequency of the feedback signal (see e.g. 102 in FIGS. 1-6) are determined. The current temperature is preferably determined when or close to when the frequency is measured (as the frequency will vary with temperature).

The frequency value may e.g. be determined by measuring or obtaining a frequency value, e.g. using a frequency counter or similar. The frequency value may e.g. by measured by measuring or counting (e.g. in reference to a known external accurate frequency) the frequency of the feedback signal, the frequency of the high-frequency reference signal, or the frequency of the high-frequency output signal (e.g. for embodiments such as shown in FIGS. 1-5). The frequency is—at least in some embodiments—measured or counted where an adjustable frequency offset circuit as disclosed herein (see e.g. 200 in FIGS. 1-6) is located or where the adjustable frequency offset circuit offsets the frequency as disclosed herein. Alternatively, the frequency may be counted (e.g. for embodiments such as shown in FIGS. 9 and 10) near the crystal-free oscillator element 120 or the LC-based oscillator (LCO) 120.

At step 904, the determined frequency value and the obtained temperature are stored in a suitable memory and/or storage for later use by the adjustable frequency offset circuit e.g. as disclosed herein.

At step 905 it is tested whether further frequency value(s) should be determined for further temperature(s). If yes, the method loops back to step 902 where the temperature is increased (or alternatively decreased) further. If no, the method ends at step 907. A number of frequency and temperature value(s) should be obtained to be sufficient to reliably cover a temperature operation range of the device that the specific crystal-free oscillator circuit is to be used in. In some embodiments, the number of frequency and temperature values is about 5 or about 4-6, but the number may vary according to specific embodiment and/or use. As an example, a temperature operation range of a device may e.g. be about 5° C. to about 50° C. or some other appropriate range depending on the specific device that the specific crystal-free oscillator circuit is to be used in.

In this way, a temperature and frequency profile is established for a particular crystal-free oscillator circuit (e.g. crystal-free oscillator element and/or PLL). It is noted, that this profile very likely (if not practically guaranteed) will be unique for the particular crystal-free oscillator circuit, crystal-free oscillator element, or PLL as these will have substantial parameter variation from circuit to circuit even when produced on a same wafer or similar. Accordingly, the parameter variation inherent for crystal-free oscillators may readily be addressed.

As mentioned, it is possible (and advantageous) to manufacture several crystal-based oscillator circuits as integrated circuits e.g. on a single wafer. In such cases, the method of FIG. 7 may comprise a repeat of steps 901-906 for a next crystal-based oscillator circuit until all relevant crystal-based oscillator circuits have been processed in this way.

This method may be fully automated and is relatively very fast. As an example, it may take less than about 90 or 100 milliseconds or even only about 10 to about 50 milliseconds to determine a profile for one crystal-based oscillator circuit.

Steps 902-904 may e.g. be carried out as explained in connection with FIG. 6, as disclosed herein, or alternatively in any other suitable manner.

It is noted that step 902 may alternatively be carried out after step 903, after step 904, or in the Y/yes branch of step 905 (then looping back to step 903 in case of yes at step 906). It is also possible to only carry out step 904 once after determining all relevant temperature and frequency values have been determined (then storing all relevant pairs in one go).

In principle and as mentioned, instead of increasing from a starting temperature, the method could be modified to decrease the temperature from a starting temperature but for many typical cases this is less practical (although initial cooling to a predetermined starting temperature before heating and measuring is practical at least for some embodiments).

FIG. 8 schematically illustrates a device, and in particular a liquid drug delivery device, comprising a crystal-free oscillator circuit for channel-based high-frequency radio communication as disclosed herein.

Shown, is an exemplary injection device 400 comprising a housing 401 encompassing various components of the injection device 400.

An arrow in FIG. 8 indicates a general distal end and a general distal direction of the injection device 400 and its components while a proximal end and direction are the opposite (the arrow points towards the distal end/in the distal direction).

The housing 401 e.g. comprises or stores a cartridge or similar where the cartridge is mounted in the housing 401 or in a cartridge holder connected to the housing 401 e.g. distally to a piston rod as in basically any or at least many types of injection devices. The cartridge may e.g. have a distal end being closed by a septum or the like and a proximal end being closed by a movable plunger or the like defining an interior of the cartridge containing a liquid drug to be expelled during use.

In some embodiments, the cartridge is replaceable while in other embodiments it is not replaceable. The latter is the case e.g. for disposable injection device, which typically involve a certain number of uses. It is typically recommended for safety reasons that such disposable injection devices are discarded after a certain period of time (e.g. about three weeks or so) even if it still contains a liquid drug to dispense.

The injection device 400 further comprises a needle cannula 402 or similar e.g. being connected to a hub or the like to form a needle assembly.

The needle cannula 402 has a distal end with a tip and a proximal end that, when the needle assembly is properly attached to the injection device 400, is in liquid communication with the interior of the cartridge.

The injection device 400 may also comprise a protective cap (not shown) surrounding at least the distal end of the needle cannula 402 and a distal end of the housing 401 when the cap is fitted or mounted to or on the housing 401.

The injection device 400 comprises a crystal-free oscillator circuit for channel-based high-frequency radio communication as disclosed herein. A crystal-free oscillator circuit for channel-based high-frequency radio communication as disclosed herein is particularly advantageous for use in or with such a disposable injection device 400 as the costs for a communications capable disposable device is reduced. In this way, it is possible to provide communications related functionality (sending/receiving information, data, etc.) even for more or less disposable products e.g. involving only a single use, a few uses, or uses only for a limited amount of time such as for about a month or couple of weeks or less.

In some embodiments, the injection device 400 is an insulin injection device or a disposable insulin injection device. An injection device of the type shown in FIG. 8 is generally also referred to a pen-based injection device.

FIG. 9 schematically illustrates an exemplary embodiment of a crystal-free oscillator element according to various embodiments. Shown is an embodiment of a crystal-free oscillator element 120 as disclosed herein and in the form of an LC oscillator receiving a bias current (I_{bias}) from a controllable current source 801 and being connected to an electrical reference potential 802 such as electrical ground. The crystal-free oscillator element 120 is as an example a differentially implemented CMOS oscillator comprising four transistors 804, connected as shown, and an LC resonator circuit 803. The LC resonator circuit 803 is tuneable with respect to frequency and comprises, in the shown embodiment, a fixed value inductor 806 (e.g. comprising one or more inductors) and a controllable and variable capacitor 805 (comprising one or more but preferably a plurality of capacitors e.g. as shown and explained further in connection with FIG. 10) connected in parallel. By varying the capacitance 805 via one or more control signals (illustrated by the “freq. tuning” signal(s)), the resonant frequency and thereby the output frequency (i.e. the high-frequency reference signal 101) of the crystal-free oscillator element 120 can controllably be adjusted. According to at least some embodiments, the frequency is tuned (e.g. as explained in connection with FIG. 10) to compensate for a temperature dependency of the crystal-free oscillator circuit or element in response to a measured current operating temperature as disclosed herein. It is noted, that in this respect the frequency adjustment here is made for temperature compensation purposes where an adjustable frequency offset circuit 210 still will offset the frequency in a feedback loop of a PLL as disclosed herein to provide the advantages associated therewith. The bias current may be adjusted to provide an optimal operating point of the crystal-free oscillator element 120 but it is not, at least not according to this and corresponding embodiments, used for frequency adjustments.

FIG. 10 schematically illustrates further details of the crystal-free oscillator element of FIG. 9 together with additional elements. Illustrated is a crystal-free oscillator element 120 being supplied with a bias current 801 and generating an oscillating output signal (F_{LCO}) 101 to a PLL 110 as disclosed herein. The crystal-free oscillator element 120 is illustrated together with a controllable and variable capacitor side 805 of an LC resonator circuit (see e.g. 803 in FIG. 9). In the shown embodiments, the controllable and variable capacitor side 805 comprises at least one fixed or base capacitor 841 (illustrated as one capacitor and labelled C1), a group of (in this particular embodiment nine) switchable capacitors 842 (illustrated as one capacitor and labelled C2), e.g. arranged in a capacitor bank or the like, and at least one voltage controlled capacitor 843 (illustrated as one capacitor and labelled C3), e.g. a varactor or the like, connected in parallel.

The group of switchable capacitors 842 is controlled in response to a first tuning control signal 830 (labelled course-tune), as an example in the form of an 9 bit digital signal (for a group of nine capacitors), controlling which of the switchable capacitors of the group 842 should be activated at any given time.

Additionally, the at least one voltage controlled capacitor 843 is controlled in response to a second tuning control signal 831 (labelled fine-tune) in the form, as an example, a 10 bit digital signal. The second tuning control signal 831 is converted into an analog voltage signal by a digital to analog converter (DAC) 820 thereby controlling the amount of voltage received by the at least one voltage controlled capacitor 843 in dependency of the second tuning control signal 831. Accordingly, the output of the crystal-free LC oscillator element 120 can be tuned coarsely by the first tuning control signal 830 and finely by the second tuning control signal 831 enabling very precise and efficient control of the frequency output by the crystal-free LC oscillator element 120.

Further shown, is a temperature sensor 610 e.g. or preferably located near the crystal-free oscillator element 120 providing a temperature dependent voltage representative of the obtained temperature where the voltage is converted, by an analog to digital converter ADC 810, into a 10 bit, as an example, digital sensor signal 832 being provided to a processing circuit or element for generation (and supply) of the first and second tuning control signals 830, 831 in dependency thereto.

Further shown is a controllable heating element 630 e.g. or preferably also located near the crystal-free oscillator element 120 that in response to a heating control signal 833 generates heat in dependency thereto. The controllable heating element 630 is at least in some embodiments a resistor circuit or element 630 generating heat in response to being provided with an electrical current. The heating control signal 833 may e.g. be supplied by the processing circuit or element (and e.g. converted from a digital signal to an analog current signal by a suitable ADC (not shown)). The controllable heating element 630 may e.g. be used as disclosed herein and in particular as disclosed in connection with FIG. 7.

Some preferred embodiments have been shown in the foregoing, but it should be stressed that the invention is not limited to these, but may be embodied in other ways within the subject matter defined in the following claims.

In the claims enumerating several features, some or all of these features may be embodied by one and the same element, component or item. The mere fact that certain measures are recited in mutually different dependent claims or described in different embodiments does not indicate that a combination of these measures cannot be used to advantage.

It should be emphasized that the term “comprises/comprising” when used in this specification is taken to specify the presence of stated features, elements, steps or components but does not preclude the presence or addition of one or more other features, elements, steps, components or groups thereof.

1. A crystal-free oscillator circuit for channel-based high-frequency radio communication, the crystal-free oscillator circuit comprising
a crystal-free oscillator element configured to provide a high-frequency reference signal, the high-frequency reference signal having a frequency of at least about 1 GHz, and
a phase-locked loop (PLL) circuit having a feedback loop and comprising a PLL oscillator, wherein the phase-locked loop circuit (110) is configured to receive the high-frequency reference signal, to provide a feedback signal in the feedback loop, and to provide a high-frequency output signal, the high-frequency output signal being generated by the PLL oscillator in response to the high-frequency reference signal and to the feedback signal where the feedback signal is dependent on an earlier instance of the high-frequency output signal,
wherein the crystal-free oscillator circuit further comprises an adjustable frequency offset circuit located in the feedback loop, the adjustable frequency offset circuit comprising a frequency generator and being configured to offset a frequency of the feedback signal in response to an adjustment control signal, and wherein the crystal-free oscillator circuit is configured to compensate for a temperature dependency of the crystal-free oscillator circuit in response to a measured current operating temperature.

2. The crystal-free oscillator circuit according to claim 1, wherein the adjustment control signal represents or comprises a frequency offset value to apply to offset the frequency of the feedback signal.

3. The crystal-free oscillator circuit according to claim 1, wherein the adjustment control signal is provided in response to
an obtained or received temperature signal representing a current operating temperature of at least a part of the crystal-free oscillator circuit, and
a predetermined relationship or function between operating temperatures of the at least a part of the crystal-free oscillator circuit and predetermined respective associated frequency offset values.

4. The crystal-free oscillator circuit according to claim 3, wherein
the predetermined relationship or function has been determined for the particular crystal-free oscillator circuit.

5. The crystal-free oscillator circuit according to claim 3, wherein the crystal-free oscillator circuit comprises a temperature sensor circuit or element configured to measure a current temperature of the at least a part of the crystal-free oscillator circuit and to provide the temperature signal in response thereto.

6. The crystal-free oscillator circuit according to claim 3, wherein the crystal-free oscillator circuit is a solid-state integrated circuit or a part thereof that further comprises a controllable heating element and a frequency counter, wherein the solid-state integrated circuit is configured to determine the predetermined relationship or function between operating temperatures and respective associated frequency values for a particular crystal-free oscillator circuit by incrementally or continuously increasing a temperature of at least a part of the crystal-free oscillator circuit using the heating element and obtaining
a number of temperature values and associated frequency values, obtained by a frequency counter at respective temperature values, or
a number of temperature values and associated frequency offset values derived from frequency target values and associated frequency values, obtained by the frequency counter at respective temperature values.

7. The crystal-free oscillator circuit according to claim 6, wherein the controllable heating element is a resistor circuit or element generating heat in response to being provided with an electrical current.

8. The crystal-free oscillator circuit according to claim 1, wherein the crystal-free oscillator circuit further comprises a first static frequency divider located in the feedback loop and being configured to divide down a frequency of the feedback signal by a factor being a first predetermined positive integer (N).

9. The crystal-free oscillator circuit according to claim 1, wherein the crystal-free oscillator circuit further comprises a second static frequency divider located in the feedback loop and being configured to divide down a frequency of the feedback signal by a factor being a second predetermined positive integer (M), and wherein the adjustable frequency offset circuit is configured to offset the frequency of the feedback signal after being divided down by the second static frequency divider.

10. The crystal-free oscillator circuit according to claim 9, wherein the frequency generator and the second static frequency divider each provide a first and a second output, and the adjustable frequency offset circuit comprises a first mixer or modulator, a second mixer or modulator, and an adding element, wherein the adjustable frequency offset circuit is configured
to mix or modulate, by the first mixer or modulator, the first output from the frequency generator and the first output of the second frequency divider resulting in a first mixed or modulated signal,
to mix or modulate, by the second mixer or modulator, the second output from the frequency generator and the second output of the second frequency divider resulting in a second mixed or modulated signal, and
to add, by the adding element, the first and the second mixed or modulated signals and supply the resulting signal as output of the adjustable frequency offset circuit.

11. The crystal-free oscillator circuit according to claim 1, wherein the crystal-free oscillator circuit further comprises
a phase frequency detector (PED) being configured to receive the high-frequency reference signal and the feedback signal and to derive at least one phase error signal in response thereto, and
a low-pass filter (LPF) being configured to low-pass filter the at least one phase error signal and to derive an oscillator input signal in response thereto, wherein the PLL oscillator element or circuit is configured to derive the high-frequency output signal in response to the oscillator input signal.

12. The crystal-free oscillator circuit according to claim 1, wherein the crystal-free oscillator element is an LC-based oscillator.

13. The crystal-free oscillator circuit according to claim 12, wherein the LC-based oscillator (LCO) comprises a fixed inductor part and a controllable and variable capacitor part (805), wherein the controllable and variable capacitor part comprises at least one fixed or base capacitor and one or more of: a group of switchable capacitors, controlled in response to a first tuning control signal, and at least one voltage controlled capacitor, controlled in response to a second tuning control signal, wherein the LC-based oscillator (LCO) is configured to be temperature compensated by adjusting an output frequency of the LC-based oscillator (120) in according with the first tuning control signal and/or the second tuning control signal provided in response to a temperature sensor signal provided by a temperature sensor located in the vicinity of the LC-based oscillator (LCO).

14. The crystal-free oscillator circuit according to claim 1, wherein the high-frequency reference signal has a frequency of about 2 GHz, or of about 2 GHz or more.

15. The crystal-free oscillator circuit according to claim 1, wherein the crystal-free oscillator circuit is implemented as a monolithic integrated circuit.

16. The crystal-free oscillator circuit according to claim 1, wherein the output signal is provided to a channel-based radio communication element or system comprising a Bluetooth or Bluetooth Low Energy communication element or system.

17. A channel-based radio communication device or system comprising a crystal-free oscillator circuit according to claim 1.

18. A method of deriving a unique temperature and frequency profile for a particular crystal-free oscillator circuit, e.g. according to claim 1, the method comprising:
determining a relationship or function between operating temperatures and respective associated frequency values of a feedback signal or a reference signal or a high-frequency output signal of the particular crystal-free oscillator circuit by incrementally or continuously increasing a temperature of at least a part of the particular crystal-free oscillator circuit using a heating element,
and obtaining, and storing in a memory and/or storage 640, a number of
temperature values and associated frequency values, obtained by a frequency counter at respective temperature values, or
temperature values and associated frequency offset values derived from frequency target values and associated frequency values, obtained by the frequency counter at respective temperature values.

19. The method according to claim 18, wherein the steps are repeated for a number of different particular crystal-free oscillator circuits being part of a same wafer.

20. A medical device comprising a crystal-free oscillator circuit according to claim 1.

21. The medical device according to claim 20, wherein the medical device is a liquid drug delivery device, e.g. an injection device for delivering set doses of a liquid drug, comprising
a housing storing, in use, a cartridge having a distal end being closed by a septum and a proximal end being closed by a movable plunger defining an interior containing the liquid drug, and
a needle cannula having a distal end with a tip and a proximal end, which proximal end is in liquid communication with the interior of the cartridge when the needle cannula and the cartridge is mounted in the liquid drug delivery device.

22. A medical device according to claim 20, wherein the medical device or the channel-based radio communication device or system is a disposable and/or a time-limited use product.
